(19) United States
(12) Patent Application Publication
(10) Pub. No.: US 2017/0266358 A1
(43) Pub. Date: Sep. 21, 2017

(54) CARDIAC CONNECTION FOR VENTRICULAR ASSIST DEVICE

(71) Applicant: EverHeart Systems Inc., Webster, TX (US)

(72) Inventor: Greg S. ABER, Houston, TX (US)

(21) Appl. No.: 15/462,541

(22) Filed: Mar. 17, 2017

Related U.S. Application Data
(60) Provisional application No. 62/310,575, filed on Mar. 18, 2016.

(51) Int. Cl. A61M 1/10; A61M 1/12

(52) U.S. Cl. CPC A61M 1/1008; A61M 1/122

(57) ABSTRACT

A cardiac support system can be used to couple a conduit of a pump to a heart. The cardiac support system can include a sewing ring configured for attachment to the heart, a connector having a ring frame attached to the sewing ring, and having a channel and a frame recess extending radially outwardly from the channel and a spring component disposed partially within the frame recess. The cardiac support system can also include a pump housing having a radial protrusion having a distal surface and a proximal surface and a housing recess extending radially inwardly from the radial protrusion and configured to receive a portion of the spring component. The housing may also include a seal to form a fluid tight barrier between the housing and ring frame of the connector.

### RELATED APPLICATIONS

This application claims the benefit of U.S. Provisional Application No. 62/310,575, filed Mar. 18, 2016, the entirety of which is hereby incorporated herein by reference.

### FIELD

The subject technology relates to connections of medical devices to patient anatomy and, in some embodiments, more particularly to connections of pumps to hearts.

### BACKGROUND

In certain disease states, the heart lacks sufficient pumping capacity to meet the needs of the body. This inadequacy can be alleviated by providing a mechanical pump referred to as a cardiac support system or a ventricular assist device (“VAD”) to supplement the pumping action of the heart. The VAD can remain in operation for months or years to keep the patient alive while the heart heals, remain in operation permanently during the patient's lifetime if the heart does not heal, or keep the patient alive until a suitable donor heart becomes available.

The VAD is typically connected to the heart, most commonly to the left ventricle. Once connected, the VAD and the heart pump blood from the left ventricle to the ascending or descending aorta to improve blood flow. Alternatively, a VAD may be connected to the ventricle to assist the heart in pumping blood to the pulmonary arteries.

The VAD can be hydraulically connected to the heart using a connector. First, the connector is attached to the outer surface of the heart, for example, using sutures. Once coupled, a surgical tool is then used to core a hole in the ventricle through the connector. An inflow housing extending from the VAD is inserted through the hole in the left ventricle. The VAD is then attached to the connector such that an inflow housing of the VAD is positioned within the central opening of the connector.

### SUMMARY

Despite the development of technology in this field, VAD ventricular connectors continue to be bulky and fail to provide ease of connection between a VAD and the ventricle. Existing systems and methods require the use of precision tools for applying torque to clamp screws or bulky latching mechanisms which take up valuable space within the pericardium.

However, in accordance with some embodiments disclosed herein, various systems and methods provide connection and disconnection of the VAD without the need for precision torque application tools or bulky latching mechanisms.

Further, in some embodiments, a VAD or cardiac support system may assist or fully support a required blood flow of a patient. For example, a pump can assist with ventricular functions of the heart. In order to provide flow of blood through a chamber (e.g., left ventricle, right ventricle, or both) of the heart, a pump should have a connection to seal and persistently attach to the heart. Some embodiments disclosed herein provide a structure that enables a surgeon to exert a force to connect to the pump that is sufficiently low to avoid damage to heart tissue during installation, while the force required to disconnect the pump is sufficiently high to avoid inadvertent disconnection of the pump after implantation.

Accordingly, in some embodiments, a cardiac support system described herein enables pump connection with a low force and no surgical tools while requiring a greater force for disconnection. The cardiac support system can thereby provide for greater patient safety and simplified surgical procedure during and after installation than does a system that requires precision torque application tools or bulky latching mechanisms. According to some embodiments, the cardiac support system can also be used with a mechanism for applying a force required to disconnect the pump in a manner that minimizes the effect of the force on the anatomy of the patient.

The cardiac support system can include a connector and a blood pump. The connector (e.g., apical ring or cuff) can serve as an interface between an inflow housing of the pump and a ventricle (e.g., left ventricle) of the heart. The connector may perform the functions of providing a suture attachment point onto epicardium muscle of the heart and sealing against potential blood leakage from the left ventricle around or outside of the inflow housing. The connector can include a spring component that can be collapsed to allow a radial protrusion of the pump to pass through the connector. The proximal and distal surfaces of the radial protrusion can be configured to collapse the spring component in different ways.

In some embodiments, the spring component can comprise a single loop of a coil spring, sections of a canted coil, or separate resilient elements positioned about a circumference of the connector for contact with the radial protrusion of the pump.

For example, a coil spring and the protrusion can be configured so that the coil spring is collapsed and the pump is received into a secure engagement within the connector when the distal surface applies a low connection force to the coil spring and allows the pump inflow housing to pass through and latch the coil spring behind the radial protrusion. The pump is retained in the secure engagement until the proximal surface applies a high disconnection force to the coil spring. The disconnection force required to remove the pump by collapsing the coil spring with the proximal surface can be greater than the connection force required to insert the pump by collapsing the coil spring with the distal surface. Accordingly, the pump can be inserted into the connector with less force than is required to remove the pump. As such, a risk of inadvertent removal of the pump from the connector is reduced.

To remove the pump from the connector, a wedge separation tool can be inserted laterally by hand at a location axially between the pump and the connector until the coil spring is collapsed with the proximal surface of the radial protrusion. A first end of the wedge separation tool can have a first thickness, and a second end of the wedge separation tool can have a second thickness, greater than the first thickness. The first end of the wedge separation tool is inserted and progresses until the second end is axially between the pump and the connector. The disconnection force is achieved gradually during insertion of the wedge separation tool. The wedge separation tool can move in a direction that is transverse to the axis along which the pump and the connector move relative to each. At least a component of the force applied by the wedge separation tool is along the same axis which the pump and the connector move relative to each other allowing disconnection. Minimal forces transverse to the axis and no torque applied to the wedge separation tool are required. Accordingly, disconnection is achieved while avoiding traumatic forces on the heart and other patient anatomy with a hand applied tool.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1A shows a perspective view of a cardiac support system with some components positioned within a patient and other components positioned outside the patient, according to some embodiments of the present disclosure.

FIG. 1B shows a perspective view of a pump and a connector of the cardiac support system of FIG. 1A, according to some embodiments of the present disclosure.

FIG. 2A shows a top view of the pump of FIG. 1B, according to some embodiments of the present disclosure.

FIG. 2B shows a sectional view of the pump of FIG. 1B, according to some embodiments of the present disclosure.

FIG. 3 shows a sectional view of the connector of the cardiac support system of FIG. 1A, according to some embodiments of the present disclosure.

FIG. 4 shows a sectional view of the pump inflow housing of the cardiac support system of FIG. 1A, according to some embodiments of the present disclosure.

FIG. 5 shows a simplified, schematic partial sectional view of the pump and the connector of the cardiac support system of FIG. 1A, according to some embodiments of the present disclosure.

FIG. 6 shows a top view of a spring component, according to some embodiments of the present disclosure.

FIG. 7 shows a top view of a portion of the spring component of FIG. 6 in relaxed and collapsed states, according to some embodiments of the present disclosure.

FIG. 8A shows a sectional view of the connector of FIG. 3 and the pump inflow housing of FIG. 4 separated from and aligned for connection to each other, according to some embodiments of the present disclosure.

FIG. 8B shows a sectional view of the pump inflow housing of FIG. 4 and the connector of FIG. 3 in the process of coupling, according to some embodiments of the present disclosure.

FIG. 8C shows a sectional view of the pump inflow housing of FIG. 4 and the connector of FIG. 3 coupled together and latched, according to some embodiments of the present disclosure.

FIG. 9A shows a perspective view of a wedge separation tool, according to some embodiments of the present disclosure.

FIG. 9B shows a side view of the wedge separation tool of FIG. 9A, according to some embodiments of the present disclosure.

FIG. 9C shows a top view of the wedge separation tool of FIG. 9A, according to some embodiments of the present disclosure.

FIG. 10A shows a partial sectional view of the pump inflow housing of FIG. 4 and the connector of FIG. 3 coupled together with the wedge separation tool of FIG. 9A, according to some embodiments of the present disclosure.

FIG. 10B shows a partial sectional view of the pump inflow housing of FIG. 4 and the connector of FIG. 3 being separated with the wedge separation tool of FIG. 9A, according to some embodiments of the present disclosure.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

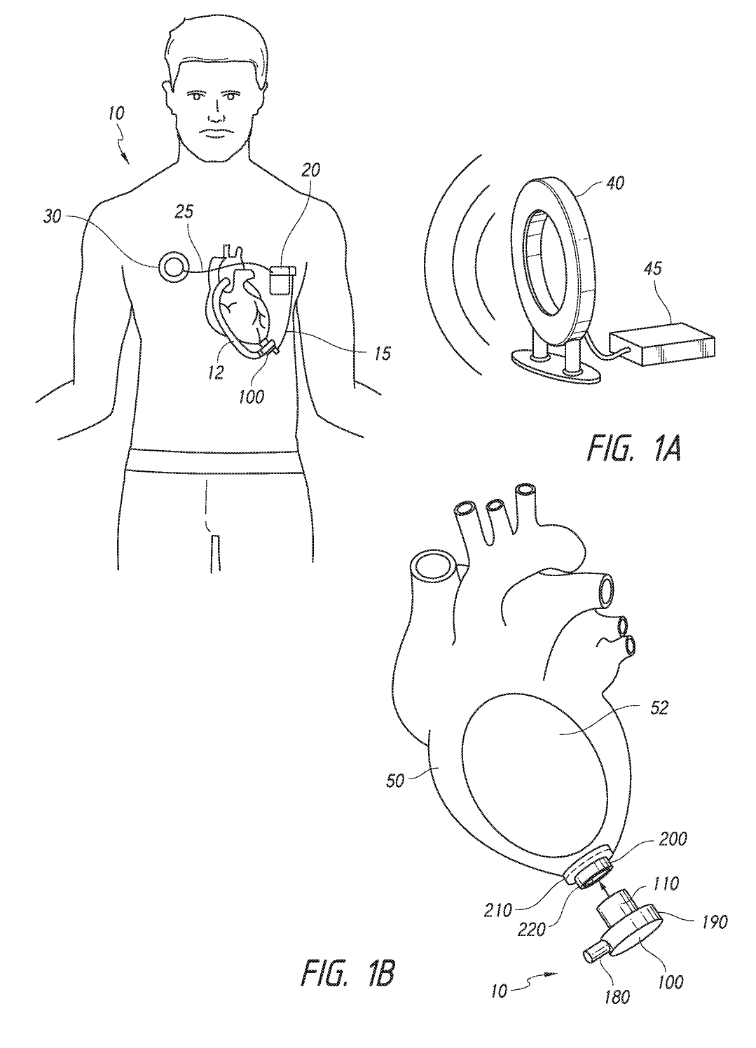

According to some embodiments, for example as shown in FIG. 1A, a cardiac support system 10 can be provided that can be used to treat a patient with a heart condition. The cardiac support system 10 can include components implanted within the patient, such as an outflow graft 12, a power module 20, a receiving coil assembly 30, and a blood pump 100. The cardiac support system 10 can further include a transmitting coil assembly 40 and a radio frequency (RF) power supply 45 outside the patient for wirelessly recharging power module 20.

During operation, the pump 100 receives power from the power module 20, which is charged from the receiving coil assembly 30. The receiving coil assembly 30 can receive power wirelessly via a coupling (e.g., magnetic resonance coupling) with the transmitting coil assembly 40. The receiving coil assembly 30 can be electrically connected to the power module 20 by a receiver cable 25. The power module 20 can be electrically connected to the pump 100 by a pump cable 15. Electrical power can be delivered to the pump 100 through the pump cable 15.

The blood pump 100 has an inlet of an inflow housing 110 and an outlet 180 connected to an impeller chamber of an impeller housing 190, within which an impeller (not shown) resides. When the impeller rotates, it imparts motive energy to a fluid in the impeller chamber to increase flow of that fluid from the inflow housing 110 to the outlet 180. When applied to the cardiac circulatory system, the increased flow may be used for therapeutic purposes such as, but not limited to, ventricular assist (right, left and both) and heart replacement.

The pump 100 can be attached to the heart to provide flow of blood through a chamber (e.g., left ventricle, right ventricle, or both) of the heart. The cardiac support system 10 comprises a connector 200 configured attach to the heart and to receive and engage the pump 100. According to some embodiments, for example as shown in FIG. 1B, the inflow housing 110 of the pump 100 can be attached to the heart via the connector 200, for example, at a left ventricular apex of the heart. The outlet 180 can be attached to the aorta via the outflow graft 12. Each of the inflow housing 110 and the outlet 180 are generally attached to the natural tissue by sutures through the use of a sewing ring or cuff, described below, so that blood flow communication is established and maintained without external leakage.

The connector 200 may be coupled to the heart through a process of elevating the left ventricle out of the pericardial sac, suturing the connector 200 at the apex, and coring through the apex inside the connector 200. The distal end of the inflow housing 110 of the pump 100 is inserted through the cored hole and into the heart in order to establish blood flow from the heart to the pump 100. The pump 100 is secured to the connector 200 as described further herein.

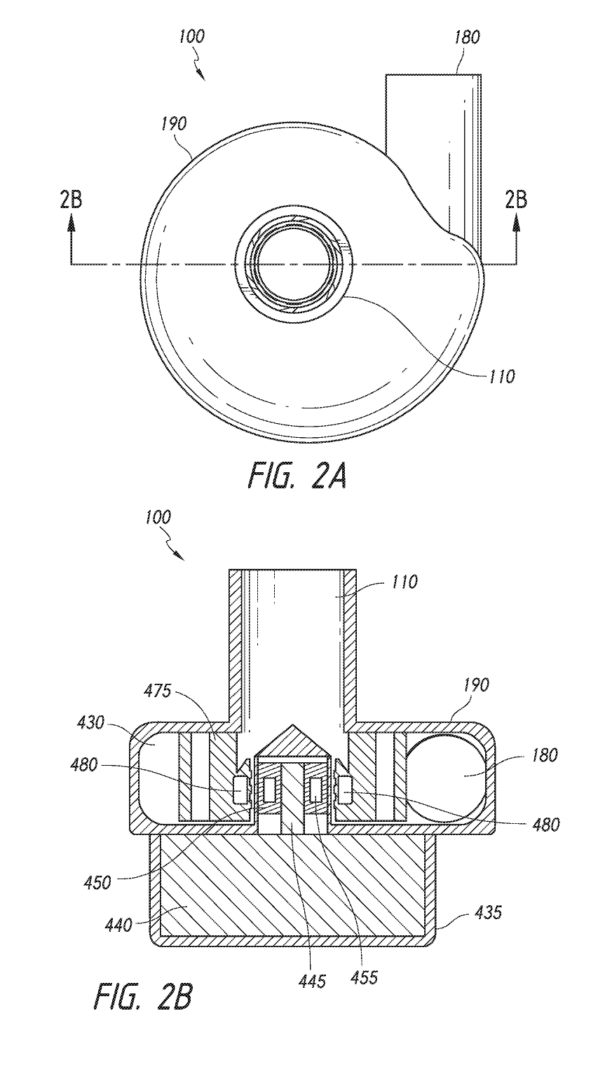

According to some embodiments, as shown in FIGS. 2A and 2B, the pump 100 can be a rotary blood pump. The pump 100 can include an impeller housing 190 providing an inflow housing 110, an outlet 180, and a motor housing 435. The impeller housing 190 can include two or more pieces and may be joined by welding. The impeller housing 190 can provide an impeller chamber 430 for an impeller 475. The impeller chamber 430 can have the inflow housing 110 for connection to a fluid source and the outlet 180 for providing fluid to a desired location. The impeller 475 can be driven by one or more of a variety of mechanisms. For example, the impeller 475 can be driven by one or more permanent magnets and/or magnetic materials 480. The permanent magnets 480 can allow impeller 475 to be magnetically coupled to a hub 450. The magnetic coupling can allow the motor 440 to cause the impeller 475 to rotate when the motor 440 rotates the hub 450.

A motor housing 435 can be attached to the impeller housing 190 to form a fluid and/or pressure tight chamber for the motor 440. While the motor housing 435 is shown as a separate component from the impeller housing 190, the impeller housing 190 and the motor housing 435 can also be combined to form a single combined housing. In particular, the motor housing 435 is shown separate from the pump 100. The motor 440 can be entirely contained between the impeller housing 190 and the motor housing 435. However, any other suitable driving means can also be utilized. The motor 440 can provide a shaft 445 with the hub 450 mounted to the shaft 445. The hub 450 can include one or more permanent magnets and/or magnetic materials 455 for magnetic coupling to the permanent magnets 480.

As the impeller 475 rotates, it can be supported by one or more bearings, such as radial bearings, axial bearings, hydrodynamic bearings, contact bearings, journal bearings, and combinations thereof. It will be appreciated that other configurations can be provided for the pump 100 for use with the connector 200.

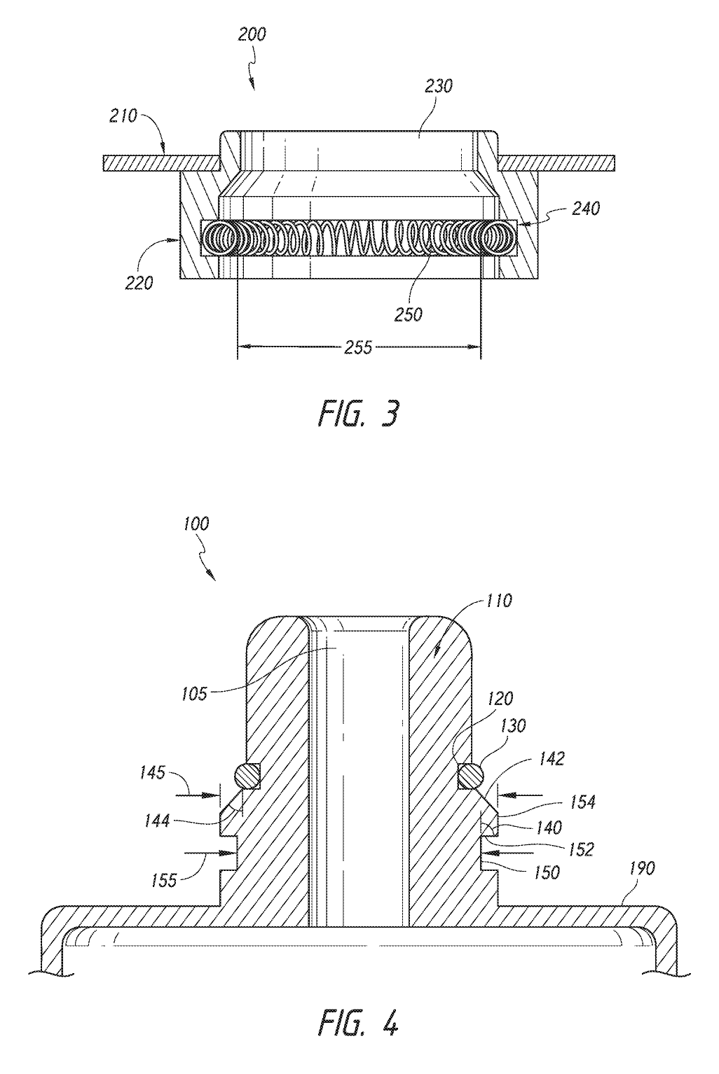

According to some embodiments, for example as shown in FIG. 3, the connector 200 can include a sewing ring 210 and a ring frame 220. The sewing ring 210 provides an interface for attachment to the natural tissue of the heart with sutures. The sewing ring 210 can be fixedly attached to the ring frame 220 also using sutures. The ring frame 220 is a rigid structure that provides a channel 230 for receiving the pump 100. The ring frame 220 can comprise a biocompatible material, such as titanium.

The ring frame 220 can comprise a portion of an engagement mechanism that enables the ring frame 220 to be coupled with another portion of the engagement mechanism on a blood pump. When used in concert, the engagement mechanism, having at least a portion thereof on the pump 100 and at least a portion thereof on the connector 200, serve to create a secure interconnection In some embodiments, the engagement mechanism can comprise a female-type connector. For example, the ring frame can be configured such that the channel 230 forms at least one frame recess 240 (e.g., groove, channel, depression), which can extend radially outwardly from an inner surface of the channel 230. The frame recess 240 can extend annularly about a longitudinal axis of the channel 230. Thus, in some embodiments, the frame recess 240 can comprise a continuous annular or circular channel that is recessed into the ring frame 220. However, in some embodiments, the frame recess 240 can comprise a plurality of discontinuous grooves, channels, or depressions that extend and a circular path about the inner surface of the channel 230.

In order to facilitate engagement with the blood pump, the engagement mechanism can comprise a spring component disposed within the frame recess 240, such as a coil spring 250. The coil spring 250 can change shape and/or dimensions to accommodate passage of a portion of the pump 100, as discussed further herein. The spring component can extend entirely or at least partially along the interior of the channel 230. For example, when using the coil spring 250 or other single-piece or continuous spring member, the coil spring 250 can extend about the entirety of the circular path of the channel 230. Additionally, in some embodiments, the coil spring 250 can have a relaxed inner cross-sectional dimension 255 when in a relaxed state within the frame recess 240. The spring component can advantageously provide the unique property of permitting engagement between the blood pump and the ring frame 220 using a lower amount of force than that required to disengage these components at least in part because of the compression of the spring component when engaged, as discussed herein.

According to some embodiments, for example, as shown in FIG. 4, the pump 100 can include the inflow housing 110 and the impeller housing 190, among other components, such as the outlet 180 (not shown in FIG. 4). The inflow housing 110 can engage with the connector 200 by using a male-type connector. This male-type connector of the inflow housing 110 can be configured to be inserted within the channel 230 of the connector 200 and engaged by the spring component (e.g., the coil spring 250) of the connector 200. When inserted, a conduit 105 (e.g., an inlet) of the inflow housing 110 can be in fluid communication with a chamber of the heart.

A seal 130 can extend annularly about the inflow housing 110 to seal at least a portion of an inner wall of the channel 230 when positioned therein. The seal 130 can comprise an O-ring or other elastically deformable structure that conforms to the inner wall of the channel 230. When the inflow housing 110 is inserted into the channel 230 of the connector 200, the engagement between the seal 130 and the inner wall of the channel 230 can prevent fluid communication across the seal 130. Alternatively or in combination with the seal 130, a seal (e.g., similar to seal 130) can be provided within a recess of the connector (e.g., the ring frame 220) for engagement with the inflow housing 110.

A radial protrusion 140 can extend radially outwardly with respect to at least a portion of the inflow housing 110. The radial protrusion 140 can have a distal surface 142 that faces in a direction toward the connector 200 when the pump 100 is inserted therein. The radial protrusion 140 can have a proximal surface 152 that faces in a direction away from the connector 200 when the pump 100 is inserted therein. The distal surface 142 of the radial protrusion 140 can be configured to gradually transition the coil spring 250 from a relaxed state to a compressed state when the inflow housing 110 is inserted into the channel 230 in a distal direction. The proximal surface 152 of the radial protrusion 140 can be configured to transition the coil spring 250 from a relaxed state to a compressed state when the inflow housing 110 is removed from the channel 230 in a proximal direction.

A housing recess 150 can extend radially inwardly from or with respect to the radial protrusion 140 on a proximal side thereof. Conversely, the radial protrusion 140 can extend radially outwardly from or with respect to the housing recess 150, whether or not the radial protrusion 140 extends radially outwardly from or with respect to any other component along the inflow housing 110. For example, while the radial protrusion 140 is shown in FIG. 4 to extend radially with respect to sections of the inflow housing 110 that are distal to the radial protrusion 140, such distal sections may be omitted, such that the radial protrusion 140 extends to a distalmost end of the inflow housing 110. The channel 230 of the connector 200 can provide a complementary shape for receiving the inflow housing 110, including the radial protrusion 140. In some embodiments, the entire length of the channel 230, other than the frame recess 240, has a consistent cross-sectional dimension (e.g., diameter). In some embodiments, the entire length of the inflow housing 110, other than the housing recess 150, has a consistent cross-sectional dimension (e.g., diameter).

The proximal surface 152 of the radial protrusion 140 can be configured to interact with the coil spring 250 in a manner that is different than that of the distal surface 142. The distal surface 142 can include a shape (e.g., frustoconical or chamfered edge) that forms a distal angle 144 (e.g., oblique angle). The distal angle 144 can be less than 90 degrees, for example, between about 15 and about 75 degrees relative to a central axis of the inflow housing 110. The proximal surface 152 can include a shape (e.g., planar or disk) that forms a proximal angle 154 (e.g., 90°) that is between about 75 and about 90 degrees relative to the central axis of the inflow housing 110. The distal angle 144 can be smaller than the proximal angle 154, such that the coil spring 250 is compressed more gradually by the distal surface 142 as the inflow housing 110 is inserted distally into the channel 230 than the coil spring is compressed by the proximal surface 152 as the inflow housing 110 is removed proximally from the channel 230.

The pump 100 can engage within the connector 200. Alternatively or in combination, the connector 200 can engage within the pump 100. According to some embodiments, the features of the pump 100 and the connector 200 can be altered to have different arrangements while operating on principles described herein. For example, while features of the connector 200, including the frame recess 240 and the coil spring 250, are shown on a radially inner surface of the channel 230, these features can also be provided on a radially outer surface of the ring frame 220. By further example, while features of the pump 100, including the seal 130, the radial protrusion 140, and the housing recess 150, are shown on a radially outer surface of the inflow housing 110, these features can also be provided on a radially inner surface of the pump 100. Accordingly, a recess (e.g., the frame recess 240 or the housing recess 150) can extend radially inwardly from an outer surface or radially outwardly from an inner surface. Likewise, a feature (e.g., the coil spring 250 or the radial protrusion 140) can extend radially outwardly from an outer surface or radially inwardly from an inner surface.

The housing recess 150 can provide corresponding engagement with the spring component of the connector 200. For example, the housing recess 150 can be configured to receive at least a portion of one or more features or aspects of the spring component in order to secure the pump 100 relative to the connector 200 along the central axis of the connector 200.

Components of the pump 100 and/or the connector 200 can include complementary shapes and cross-sectional profiles to accommodate insertion and engagement. For example, the channel 230, the frame recess 240, and the coil spring 250 of the connector 200 can be round, oval, polygonal, or another shape. By further example, the seal 130, the radial protrusion 140, and the housing recess 150 of the pump 100 can be round, oval, polygonal, or another shape. Where these components are round, any rotational orientation between the pump 100 and the connector 200 can allow insertion and engagement. Where these components are round, a degree of rotational movement between the pump 100 and the connector 200 may be permitted so that torque applied to the pump 100 is not necessarily transmitted to the connector 200 and the heart. To prevent rotation of pump 100 relative to connector 200, inflow housing 110 and channel 230 of connector 200 may have a non-circular shape such as an oval or square so that relative rotation is limited. Alternately, inflow housing 110 and channel 230 of connector 200 may have interlocking features such as splines or teeth to prevent relative rotation. In either embodiment, this may be advantageous for pump position stability after implant by reducing the chance of outflow graft 12 becoming kinked or twisted.

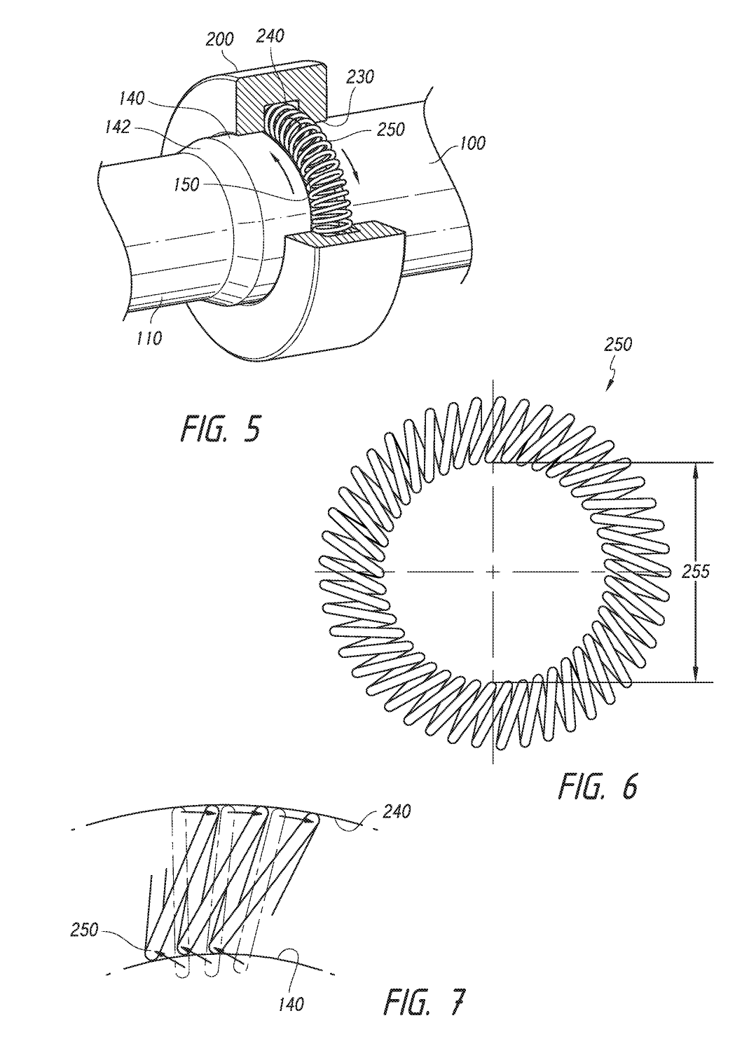

The outer cross-sectional dimension 155 of the housing recess 150 can be smaller than an outer cross-sectional dimension 145 of the radial protrusion 140. Additionally, the outer cross-sectional dimension 155 of the housing recess 150 can be smaller than the relaxed inner cross-sectional dimension 255 of the coil spring 250. According to some embodiments, for example as shown in FIG. 5, which is a simplified, schematic illustration of the pump 100 and the connector 200 shown in FIGS. 3 and 4, the coil spring 250 is permitted to recover its relaxed state when the housing recess 150 is axially aligned to be radially across from the coil spring 250 and when the coil spring 250 resides partially within the housing recess 150 and partially within the frame recess 240.

According to some embodiments, for example as shown in FIG. 6, the coil spring 250 can comprise a plurality of windings generally conforming to a shape of a torus. The coil spring 250 can be canted, such that each winding lies approximately within a plane that does not pass through the central axis of the coil spring 250. This allows the coil spring 250 to radially expand its inner cross-sectional dimension 255 when the windings collapse or “cant” further.

As shown in FIG. 7, the canted coil spring 250 yields to a force from the radial protrusion 140 on a radially inner side of the canted coil spring 250 while being pressed against the frame recess 240 on its radially outer side. In response to such a force, the windings change their respective orientations and cant further. The wire used to form the canted coil spring 250 may be a wire of a single metal or metal alloy. Alternatively, the wire may be a multi-layered wire, such as having a core of one metallic material an outer layer of another metallic material. The wire can also be a hollow wire and can have a single metal or be a multi-metallic wire with a hollow center.

According to some embodiments, the coil spring 250 need not be canted. For example, rather than deflecting each winding in a canting motion, the coil spring 250 can enlarge the inner cross-sectional dimension 255 by compressing radially. An elastic feature of the coil spring 250 can allow the windings to deform without changing orientation. For example, as the radial protrusion 140 applies a force to the coil spring 250, the windings can transition from a generally circular shape to a generally oval shape, with a dimension of the windings being reduced in a radial direction (e.g., away from the central axis of the coil spring 250).

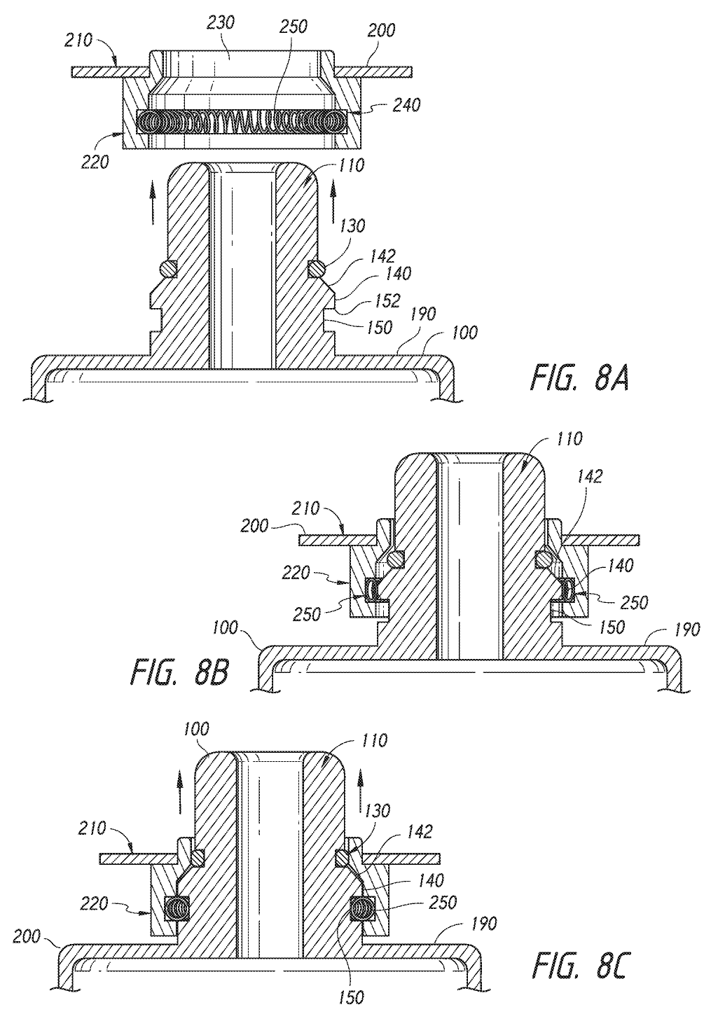

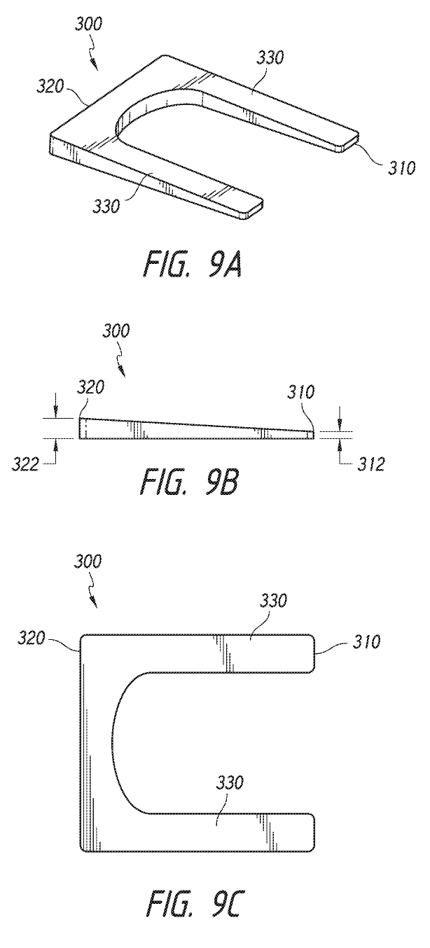

According to some embodiments, for example as shown in FIGS. 8A-8C, the inflow housing 110 of the pump 100 can be inserted into the channel 230 of the connector 200 and engaged therein. As shown in FIGS. 8A and 8B, the inflow housing 110 is inserted into the channel 230 until the distal surface 142 of the radial protrusion 140 contacts the coil spring 250. A connection force is applied to the coil spring 250 by the distal surface 142 to collapse the coil spring 250 until an inner cross-sectional dimension of the coil spring 250 is large enough to receive the radial protrusion 140. As shown in FIGS. 8B and 8C, the inflow housing 110 is further advanced into the channel 230 until the housing recess 150 is axially aligned to be radially across from the coil spring 250. At this stage, the coil spring 250 is permitted to return to a relaxed state and reside at least partially within the housing recess 150. At this stage or prior to it, the seal 130 can engage an inner wall of the channel 230. When the inflow housing 110 is fully engaged within the channel 230, an axial gap 290 can remain between the ring frame 220 and the impeller housing 190.

When engaged, the inflow housing 110 requires a particular disconnection force to be removed from the channel 230. In particular, at least the particular disconnection force must be provided to collapse the coil spring 250 with the proximal surface 152. The connection force that is required to collapse the coil spring 250 with the distal surface 142 is less than the disconnection force required to collapse the coil spring 250 with the proximal surface 152. Accordingly, the inflow housing 110 can be inserted and engaged within the channel 230 with relatively lower forces and greater ease. Furthermore, inadvertent or premature disengagement of the inflow housing 110 from the channel 230 is avoided by requiring a relatively large disconnection force. The disconnection force can be larger than forces exerted due to fluid pressure within the pump or occurring spontaneously during routine or expected activities of the patient.

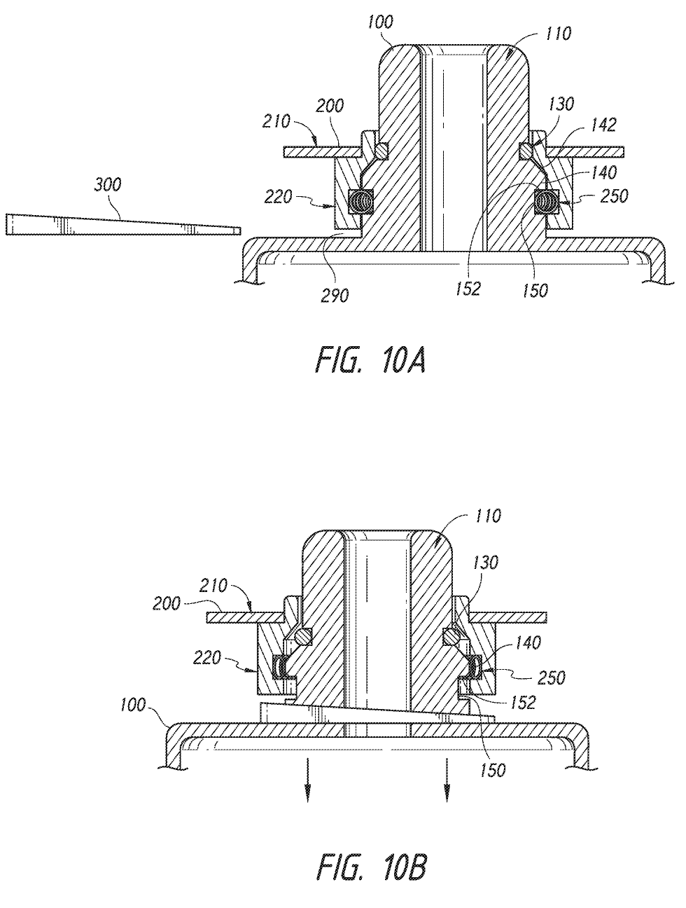

According to some embodiments, for example as shown in FIGS. 10A-B, a wedge separation tool 300 can be used to remove the inflow housing 110 from the channel 230. The wedge separation tool 300 can allow a user to apply the required disconnection force on the coil spring 250 by applying smaller forces by hand via the wedge separation tool 300 employing the mechanical force multiplying effect of a wedge. The wedge separation tool 300 can include a first end 310 having a first thickness 312 and a second end 320 having a second thickness 322, greater than the first thickness 312. The first thickness 312 can be smaller than the axial gap 290 between the ring frame 220 and the impeller housing 190. The second thickness 322 can be larger than the axial gap 290 for example, such that if the second thickness 322 of the tool were interposed within the axial gap 290, the inflow housing 110 would be disengaged from the channel 230. The thickness between the first end 310 and the second and 320 can gradually increase from the first thickness 312 to the second thickness 322. One or more arms 330 can extend between the first end 310 and the second end 320. A space between the arms 330 can receive a portion of the inflow housing 110 and/or the ring frame 220.

According to some embodiments, for example as shown in FIGS. 10A and 10B, the wedge separation tool 300 can be inserted into a region between a portion of the pump 100 and a portion of the connector 200. For example, the wedge separation tool 300 can be inserted into an axial gap 290 between the ring frame 220 and the impeller housing 190. The wedge separation tool 300 can be advanced in a direction that is generally lateral or transverse to the longitudinal axis of the inflow housing 110 and/or the channel 230. As the wedge separation tool 300 is advanced, the coil spring 250 is collapsed or compressed by virtue of its contact with the proximal surface 152 of the radial protrusion 140 until the coil spring 250 is radially opposed by the outer surface of the radial protrusion 140. Once the radial protrusion 140 is axially aligned with or radially across from the coil spring 250, further removal of the inflow housing 110 from the channel 230 requires substantially less force, which can be easily accomplished by hand.

As described, lateral advancement of the wedge separation tool 300 causes the pump 100 and the connector 200 to move away from each other as the increasing thickness between the first end 310 and the second end 320 of the wedge separation tool 300 is progressively positioned between the pump 100 and a portion of the connector 200. The lateral advancement is achieved with a generally lateral force applied to the wedge separation tool. The generally lateral force is translated into relative axial forces between the pump 100 and the connector 200. The mechanical advantage of a wedge is given by the ratio of the length of its slope to its height. Due to a slope or incline of the wedge separation tool 300, the axial force is gradually applied during lateral advancement of the wedge separation tool 300. An amount of lateral movement of the wedge separation tool 300 is larger than an amount of relative axial movement of the pump 100 and the connector 200 that results from the lateral movement. Thus, a lateral force applied directly to the wedge separation tool 300 is smaller than the disconnection force required to collapse the coil spring 250 with the proximal surface 152.

During use of the wedge separation tool 300 to separate the pump 100 and the connector 200, the force of the wedge separation tool 300 can be counterbalanced by an opposing force by a user on an opposite side of the pump 100 and/or the connector 200. Alternatively or in combination, a second wedge separation tool can be advanced in an opposite direction from an opposite side to be positioned between the pump 100 and the connector 200. The opposing forces generate a small net lateral force or no net lateral force on the pump 100 and the connector 200. Furthermore, only a small force or no force is applied to the interface between the connector 200 and the heart, and no torque along the longitudinal axis of the inflow housing 110 and/or the channel 230 is required.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A cardiac support system comprising: a connector comprising: a ring frame having a channel and a frame recess extending radially from the channel; and a spring component disposed at least partially within the frame recess; and a pump housing comprising: a radial protrusion; and a housing recess configured to receive a portion of the spring component.

Clause 2. The cardiac support system of Clause 1, wherein the housing recess has a cross-sectional dimension that is different than a cross-sectional dimension of the radial protrusion.

Clause 3. The cardiac support system of Clause 1, wherein the connector further comprises a sewing ring, coupled to the ring frame, configured to attach the connector to a heart.

Clause 4. The cardiac support system of Clause 1, wherein the radial protrusion is positioned axially between the housing recess and a distal end of the pump housing, the distal end being configured for insertion into the connector.

Clause 5. The cardiac support system of Clause 1, wherein the housing recess extends radially inwardly from the radial protrusion.

Clause 6. The cardiac support system of Clause 1, wherein the frame recess extends radially outwardly from the channel.

Clause 7. The cardiac support system of Clause 1, wherein the radial protrusion comprises a distal surface and a proximal surface.

Clause 8. The cardiac support system of Clause 7, wherein the distal surface forms a frustoconical shape.

Clause 9. The cardiac support system of Clause 7, wherein the proximal surface forms a planar shape.

Clause 10. The cardiac support system of Clause 7, wherein the proximal surface forms a proximal angle with respect to a central axis of the pump housing, and the distal surface forms a distal angle with respect to the central axis of the pump housing, the distal angle being smaller than the proximal angle.

Clause 11. The cardiac support system of Clause 7, wherein a connection force sufficient to collapse the spring component with the distal surface is less than a disconnection force required to collapse the spring component with the proximal surface.

Clause 12. The cardiac support system of Clause 1, further comprising a seal configured to form a fluid tight seal with the ring frame.

Clause 13. A kit comprising: the cardiac support system of Clause 1; and a wedge separation tool having a first end with first thickness and a second end with a second thickness, greater than the first thickness.

Clause 14. The kit of Clause 13, wherein, when the connector and the pump housing are coupled with the spring component disposed partially within each of the frame recess and the housing recess, a space between a proximally facing surface of the connector and an opposing surface of the pump housing has an axial dimension equal to or greater than the first thickness and less than the second thickness.

Clause 15. A method for coupling a conduit of a pump to a connector attached to a heart, the method comprising: inserting a male portion of the pump into a channel of the connector until a distal surface of a radial protrusion of the pump contacts a spring component within a frame recess of the connector; applying to the spring component a connection force sufficient to collapse the spring component with the distal surface to receive the radial protrusion of the pump within the spring component; and advancing the pump relative to the connector until the spring component expands into a housing recess of the pump.

Clause 16. The method of Clause 15, wherein the housing recess extends radially inwardly from the radial protrusion.

Clause 17. The method of Clause 15, wherein the frame recess extends radially outwardly from the channel.

Clause 18. The method of Clause 15, wherein the connection force is less than a disconnection force required to collapse the spring component with a proximal surface of the radial protrusion to receive the radial protrusion of the pump within the spring component.

Clause 19. The method of Clause 15, further comprising applying to the spring component a disconnection force, greater than the connection force, sufficient to collapse the spring component with a proximal surface of the radial protrusion to receive the radial protrusion of the pump within the spring component.

Clause 20. The method of Clause 15, further comprising inserting a wedge separation tool axially between the pump and the connector until the spring component is collapsed with a proximal surface of the radial protrusion to receive the radial protrusion of the pump within the spring component.

Clause 21. The method of Clause 15, further comprising inserting, on opposing radial sides of a central axis of the pump housing, two wedge separation tools axially between the pump and the connector without applying a torque or net radial force on the connector.

Clause 22. A connector of a cardiac support system, the connector comprising: a ring frame having a channel for receiving a portion of a pump housing, the ring frame having a frame recess extending radially from the channel; and a spring component disposed at least partially within the frame recess, wherein the spring component is configured to be collapsed when engaged by a radial protrusion of the pump housing, wherein the spring component is configured to expand into a housing recess of the pump housing when aligned with the housing recess.

Clause 23. A pump housing of a cardiac support system, the pump housing comprising: a radial protrusion; and a housing recess configured to receive a portion of a spring component of a connector, the housing recess having a cross-sectional dimension that is different than a cross-sectional dimension of the radial protrusion.

Clause 24. A method for disconnecting a conduit of a pump to a connector attached to a heart, the method comprising: while a spring component of the connector is expanded into a housing recess of the pump, applying to the spring component a disconnection force, greater than a connection force sufficient to collapse the spring component with a distal surface of a radial protrusion of the pump, sufficient to collapse the spring component with a proximal surface of the radial protrusion of the pump to receive the radial protrusion of the pump within the spring component.

### Further Considerations

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top” and the like as used in this disclosure should be understood as referring to an arbitrary frame of reference, rather than to the ordinary gravitational frame of reference. Thus, a top surface may extend or face upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A cardiac support system comprising:
a connector comprising:
a ring frame having a channel and a frame recess extending radially from the channel; and
a spring component disposed at least partially within the frame recess; and
a pump housing comprising:
a radial protrusion; and
a housing recess configured to receive a portion of the spring component.

2. The cardiac support system of claim 1, wherein the housing recess has a cross-sectional dimension that is different than a cross-sectional dimension of the radial protrusion.

3. The cardiac support system of claim 1, wherein the connector further comprises a sewing ring, coupled to the ring frame, configured to attach the connector to a heart.

4. The cardiac support system of claim 1, wherein the radial protrusion is positioned axially between the housing recess and a distal end of the pump housing, the distal end being configured for insertion into the connector.

5. The cardiac support system of claim 1, wherein the housing recess extends radially inwardly from the radial protrusion.

6. The cardiac support system of claim 1, wherein the frame recess extends radially outwardly from the channel.

7. The cardiac support system of claim 1, wherein the radial protrusion comprises a distal surface and a proximal surface.

8. The cardiac support system of claim 7, wherein the distal surface forms a frustoconical shape.

9. The cardiac support system of claim 7, wherein the proximal surface forms a planar shape.

10. The cardiac support system of claim 7, wherein the proximal surface forms a proximal angle with respect to a central axis of the pump housing, and the distal surface forms a distal angle with respect to the central axis of the pump housing, the distal angle being smaller than the proximal angle.

11. The cardiac support system of claim 7, wherein a connection force sufficient to collapse the spring component with the distal surface is less than a disconnection force required to collapse the spring component with the proximal surface.

12. The cardiac support system of claim 1, further comprising a seal configured to form a fluid tight seal with the ring frame.

13. A kit comprising:
the cardiac support system of claim 1; and
a wedge separation tool having a first end with first thickness and a second end with a second thickness, greater than the first thickness.

14. The kit of claim 13, wherein, when the connector and the pump housing are coupled with the spring component disposed partially within each of the frame recess and the housing recess, a space between a proximally facing surface of the connector and an opposing surface of the pump housing has an axial dimension equal to or greater than the first thickness and less than the second thickness.

15. A method for coupling a conduit of a pump to a connector attached to a heart, the method comprising:
inserting a male portion of the pump into a channel of the connector until a distal surface of a radial protrusion of the pump contacts a spring component within a frame recess of the connector;
applying to the spring component a connection force sufficient to collapse the spring component with the distal surface to receive the radial protrusion of the pump within the spring component; and
advancing the pump relative to the connector until the spring component expands into a housing recess of the pump.

16. The method of claim 15, wherein the housing recess extends radially inwardly from the radial protrusion.

17. The method of claim 15, wherein the frame recess extends radially outwardly from the channel.

18. The method of claim 15, wherein the connection force is less than a disconnection force required to collapse the spring component with a proximal surface of the radial protrusion to receive the radial protrusion of the pump within the spring component.

19. The method of claim 15, further comprising applying to the spring component a disconnection force, greater than the connection force, sufficient to collapse the spring component with a proximal surface of the radial protrusion to receive the radial protrusion of the pump within the spring component.

20. The method of claim 15, further comprising inserting a wedge separation tool axially between the pump and the connector until the spring component is collapsed with a proximal surface of the radial protrusion to receive the radial protrusion of the pump within the spring component.
